(19) United States
(12) Patent Application Publication
(10) Pub. No.: US 2021/0146060 A1
(43) Pub. Date: May 20, 2021

(54) A TORSION SPRING DRIVEN INJECTION DEVICE

(71) Applicant: Novo Nordisk A/S, Bagsvaerd (DK)

(72) Inventors: Martin Johst Christensen, Copenhagen OE (DK); Bjoern Gullak Larsen, Birkeroed (DK); Carsten Soerensen, Frederiksberg (DK); Kurt Solgaard, Graested (DK)

(21) Appl. No.: 16/623,514

(22) PCT Filed: Jun. 19, 2018
(86) PCT No.: PCT/EP2018/066236

§ 371 Date: Dec. 17, 2019

(30) Foreign Application Priority Data
Jun. 27, 2017 (EP) 17178060.4

(51) Int. Cl. A61M 5/315; A61M 5/20

(52) U.S. Cl. CPC A61M 5/31553; A61M 5/20; A61M 5/31563

(57) ABSTRACT

The invention relates to a torsion spring driven injection device for delivering set doses of a liquid drug wherein a torsion spring is strained by rotation of a rotatable dose setting structure and released by proximal movement of a piston rod driver. The dose setting structure is coupled to a drive element which is rotated during dose setting and which drive element is released during dosing to thereby rotate the piston rod driver. The piston rod driver is further coupled to the piston rod to generate a rotation and thus forward movement of the piston rod. The drive element and the piston rod driver are coupled to each other by a toothed engagement which is functional both during dose setting and during dose expelling.

### THE TECHNICAL FIELD OF THE INVENTION

The present invention relates to a torsion spring driven injection device for delivering set doses of a liquid drug. The present invention especially relates to the mechanism by which the torque of the torsion spring is stored, released and transferred to an axial movement of the piston rod. More specifically the present invention relates to such mechanism wherein the torque stored in the torsion spring is released by moving an element such as a needle shield or the like in a proximal direction upon performing the injection.

### DESCRIPTION OF RELATED ART

Generally the ejection mechanism in an injection device comprises a piston rod which moves a rubber plunger forward inside a cartridge in order to press out a set volume of liquid drug from the cartridge. The distance that the piston rod moves forward during one single injection is set and controlled by the dose setting and injection mechanism. The cartridge is often secured in a housing and the piston rod is often provided with a helical thread on the outer surface which is engaged by a nut being stationary in the housing. When the piston rod is rotated it is thus screwed forward relatively to the nut and to the housing a distance depending on the size of the set dose. Very much like an ordinary screw and nut connection wherein the nut is maintained stationary. In order to rotate the piston rod, a longitudinal track is often provided in the piston rod. This track is then engaged by a rotatable member, often referred to as a piston rod driver. Whenever this piston rod driver is rotated, the piston rod rotates simultaneously with it and is thus screwed forward relatively to the nut and henceforth relatively to the housing holding the cartridge.

The rotation of the piston rod driver can either be generated by a mechanism transforming an axial movement performed by the user pressing a part of the injection device to a rotational movement of the piston rod driver, or it can be generated by a spring which automatically rotates the piston rod driver when the force stored in the spring is released. In many known constructions as e.g. disclosed in U.S. Pat. No. 7,686,786 and in U.S. Pat. No. 8,684,969, the spring generating the rotation is a torsion spring which is strained when the user sets the size of the dose and wherein the torque so stored in the torsion spring is fully or partly released during dosing to generate a rotation of the piston rod driver.

An example of such torsion spring driven injection device is disclosed in WO16/041883. During dose setting the piston rod driver (“55” in the figures) is keyed to the housing and thus maintained inrotatable. The injection device disclosed is a so-called shield triggered injection device i.e. an injection device wherein the set dose is released when a needle shield is moved proximally. When the user pushes the needle shield against the skin to perform an injection, the proximal end of the needle shield moves the piston rod driver axially out of engagement with the housing and into engagement with the drive arrangement as depicted in FIG. 9. Both the engagement with the housing and the engagement with the drive arrangement involve a number of axially extending teeth that needs to engage. Once the injection is done and the user removes the needle shield from the skin, the piston rod driver slides out of the engagement with the drive arrangement and back into engagement with the housing. It is a prerequisite for such an injection device to work properly that the different toothed engagements are fully aligned during engagement as the engagements operates purely axially.

A similar torsion spring drive injection device is disclosed in International application PCT/EP2017/052198 (published as WO 2017/134131). This injection device operates with an axially slidable clutch (“60, 160, 260” in the figures). During dose setting this clutch rotates with the remaining dose setting element. The mechanism disclosed is also a shield triggered mechanism and the needle shield operates the clutch to move proximally during injection. When the clutch is moved proximally it is moved out of engagement with the dose setting element and into engagement with the piston rod driver such that the torsion spring rotates the clutch and therewith the piston rod driver. The different engagements here are also based on axially extending toothed engagements that needs to be properly aligned in order to engage.

A common feature for these injection devices is that an axially toothed engagement has to be established between the piston rod driver and the drive mechanism in order to transfer the torque of the torsion spring to a rotation of the piston rod driver.

If the toothed engagements are not properly aligned for engagement there is a risk that the set dose cannot be properly released or that the injection device will jam and malfunction. Further, should a torque be present in one of the involved parts, the parts will apply a rotational pressure onto each other which could obstruct the relative axial movement between the involved parts.

### DESCRIPTION OF THE INVENTION

It is an object of the present invention to provide an injection device in which the risk of misalignment of the toothed engagements and the consequences thereof are minimized and preferably eliminated.

Accordingly, in one aspect, the present invention relates to a torsion spring driven injection device for delivering set doses of a liquid drug. In such injection devices a torsion spring is rotationally strained by the user in accordance with the size of the set dose which is set by the user. The injection comprises;
A housing preferably having a longitudinal and tubular body with a longitudinal centre axis. Although such housings usually are pen-shaped other shapes could be envisaged.
A dose setting structure which is rotatable by the user in a first rotational direction relative to the housing for setting the size of the individual dose to be ejected, the rotation thus strains a torsion spring. The dose setting structure could be made from any number of individual parts but preferably comprises at least a dose setting button which is the physical part actually operated by the user.
A piston rod with an outer surface having an outer thread which extend helically in a longitudinal direction and which outer surface further is provided with a longitudinal extending engagement surface such that the outer surface of the piston rod has a non-circular cross sectional shape. The longitudinal extending engagement surface can in one example be a longitudinal groove in the piston rod or alternatively a longitudinal ridge on the outer surface of the piston rod.
A nut member which has an inner thread mating the outer thread of the piston rod. By mating is herein meant that the piston rod can be screwed helically in the threaded connection with the nut member. The nut member is preferably maintained stationary in relation to the housing. In one example the nut member is click-fitted into the housing and thereby both axially and rotationally locked to the housing, alternatively the nut member is moulded as an integral part of the housing.
A rotatable piston rod driver which engages the longitudinal extending engagement surface of the non-circular cross-section of the piston rod. By engages is herein meant that the piston rod driver is keyed to the piston rod such that the two elements rotate in unison but are able to slide axially in relation to each other. The piston rod is thus rotated to move helically when the piston rod driver is rotated relatively to the housing carrying the nut member.

Kinematic reversal is possible such that the nut member stationary in the housing is keyed to the piston rod and the piston rod driver carries the thread for the piston rod. In such an embodiment, the piston rod is moved forward without rotation in relation to the nut member.
A drive element coupled to rotationally follow the rotation of the dose setting structure in the first rotational direction during dose setting.
A torsion spring which is located between the housing and the drive element, such that a torque is built up in the torsion spring during simultaneous rotation of the dose setting structure and the drive element in the first direction. The torque is releasable to rotate the drive element in a second direction during ejection of the set dose, the second direction being rotational opposite to the first direction.

According to the invention the drive element rotates relatively to the piston rod driver during dose setting and the drive element and the piston rod driver rotate in unison during ejection of the set dose and a ratchet engagement is provided between the drive element and the piston rod driver.

Further, this ratchet engagement comprises a first set of teeth provided on the drive element engaging a second set of teeth provided on the piston rod driver and wherein the drive element and the piston rod driver are urged into engagement by a spring bias.

The spring bias thus applies an axial force onto the drive element which is urged in the distal direction into engagement with the piston rod driver. The spring bias applied by a holding spring thus holds the ratchet in engagement and the drive element is moved in the proximal direction against a spring bias upon rotation of the dose setting structure in the first direction to thereby set the size of the dose to be ejected

Since the toothing is automatically engaged during dose expelling any misalignment of the toothed engagement at least during dosing is prevented.

All though the teeth of the drive element and of the piston rod driver rides over each other during dose setting the toothed engagement is urged together by the spring bias.

The piston rod driver is preferably locked to housing during dose setting such that the first set of teeth on the drive element rides over the second set of teeth on the piston rod driver. In order to facilitate the engagement between the two set of teeth a holding spring is provided such that an axial force is applied to the drive element urging the engagement into contact after the toothing has moved one tooth in the engagement. The holding spring is in one embodiment an axial component provided in the torsion spring. Thus only one spring is needed as the torsion spring applies both a torsional force and the axial spring bias urging the drive element in the distal direction.

To further enhance the engagement, the teeth in the first set and the teeth in the second set are V-shaped with an axial flange extending substantially parallel to the centre axis and a sloped flange forming an angle to the centre axis.

The sloped flange of the teeth on the drive element thus rides over the sloped flange of the teeth on the piston rod driver during dose setting against the bias of the spring.

The steep flanges of the toothed engagement secures that the drive element is not rotated by the torsion spring. In this way the torque built up in the torsion spring during dose setting is secured but also able to be incremental reduced in accordance with the number of teeth in the toothed engagement.

During the resetting of a set dose the user rotate the dose setting structure in the opposite direction which moves the drive element in the proximal direction against a spring bias.

The second direction is thus the rotational direction used both for resetting a previously set dose and for expelling the set dose

In order to execute the resetting of the set dose, the dose setting structure comprises a dose setting button and a ratchet tube wherein means are provided between the dose setting button and the ratchet tube such that rotation of the dose setting button in the second direction moves the ratchet tube in the proximal direction.

In one example, these means are provided as wedge-shaped protrusions located on the dose setting button or on the ratchet tube and having a lifting flange with a shape which forces the ratchet tube to be lifted in the proximal direction upon rotation of the of the dose setting button in the second direction.

The ratchet tube is preferably coupled to the drive element such that the drive element moves together with the ratchet tube at least in the proximal direction thus releasing the engagement between the first set of teeth provided on the drive element and the second set of teeth provided on the piston rod driver. In that respect the height of the wedge-shaped protrusions is preferably such that the ratchet tube and the drive element is able to be lifted out of engagement with the piston rod driver.

Once the drive element is lifted out this engagement, the torsion spring is able to rotate the drive element in the opposite direction. However, the axial component of the holding spring pushes the drive element forward in the distal direction and into engagement with the previous teeth of the toothed engagement.

As in a traditional torsion spring driven injection device as sold by the Danish company Novo Nordisk A/S under the trade name FlexTouch®, the ratchet tube can be permanently coupled to the drive element either by a click fit or by moulding the parts together. However, alternatively the ratchet tube can engage the drive element by a toothed engagement.

In details the ratchet tube is distally provided with a plurality of V-shaped teeth, and the drive element has an inner ring of V-shaped teeth, and wherein the V-shaped teeth on the ratchet tube is positioned such in relation to the teeth on the drive element that the ratchet tube is rotational in relation to the drive element in the second direction.

In one example, an End-of-Content counter is provided between the piston rod and the ratchet tube, however since the ratchet tube is able to rotate relatively to the drive element it is possible to rotate the ratchet tube and the piston rod together whereby the EoC counter remains in its position on the piston rod. It is henceforth possible to rotate the piston rod into engagement with the plunger inside the cartridge without changing the distance between the EoC counter and a stop flange provided proximally on the piston rod. It is therefore possible to maintain the correlated injectable volume as a constant all though the piston rod is rotated in the distal direction.

In order to rotate the piston rod into such “zero point adjustment”, the piston rod driver must be released from the housing to thereby rotate together with the piston rod. Also if a piston rod foot is provided distally on the piston rod, this piston rod foot is the element abutting the plunger inside the cartridge once the correct position of the piston rod has been obtained.

During this zero-point adjustment, the scale drum which is positioned in its zero position hinders the drive element from rotation with the result that the torsion spring is not strained during the zero point adjustment.

In all examples it is preferred that the torsion spring is pre-strained during assembly such that a small torque is present in the torsion spring even with the scale drum being in its zero position. This secures that sufficient torque is present even at small dose settings such that frictions can be overcome.

After the individual dose to be expelled has been set by the user by rotation of the dose setting structure and the thereby straining of the torsion spring, the set dose is expelled by moving the piston rod driver proximally in relation to the housing to a rotationally unlocked position out of engagement with the housing. This will course the teeth on the drive element to engage with the teeth on the piston rod driver and since the piston rod driver is no longer prevented from rotation the torque of the torsion spring will force the piston rod driver to rotate.

Since the piston rod driver is keyed to the piston rod this will simultaneously generate a rotation of the piston rod whereby the piston rod will be moved distally and the set dose will be expelled.

In one example, the piston rod driver is moved proximally in relation to the housing and out of engagement with the housing by a release trigger. Such release trigger can in one example be coupled to a slider provided on the outer surface of the housing such that the release trigger is moved axially whenever the user operates the slider. In a different example, the release trigger can be coupled to a needle shield such that an axial movement of the needle shield moves the release trigger in the axial direction. This is often referred to as a shield triggered release and is often used such that the set dose is released when the user presses the needle shield against the skin.

The needle shield is usually made such that the needle cannula is hidden for the user during use. The only action required by the user is thus to push the needle shield against the skin which triggers the injection where after the user can remove the needle shield from the skin. Typically a compression spring urges the needle shield back to its initial position covering the needle cannula.

In details the torque of the torsion spring rotates the drive element during ejection of the set dose such that the rotational force is transferred from the axial flanges of the teeth on the drive element to the axial flanges on the teeth on piston rod driver thereby creating a rotation of the piston rod driver.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and connected to a hub to form a complete “injection needle” also often referred to as a “needle assembly”. A needle cannula could however also be made from a polymeric material or a glass material. The hub also carries the connecting means for connecting the needle assembly to an injection apparatus and is usually moulded from a suitable thermoplastic material.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug.

The cartridges used for both pre-filled injection devices and for durable injections devices are typically factory filled by the manufacturer with a predetermined volume of a liquid drug. A large number of the cartridges currently available contains either 1.5 ml or 3 ml of liquid drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled injection device” or “Disposable injection device” is meant an injection device containing a predetermined quantum of a liquid drug and which injection device is disposed of once this predetermined quantum has been used. The cartridge containing the liquid drug is permanently positioned or embedded in the injection device such that the user cannot remove the cartridge without permanent destruction of the injection device. Once the predetermined amount of liquid drug in the cartridge and thus in the injection device is used either in one injection or in a series of multiple injections, the user discards the entire injection device including the embedded cartridge.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” or “permanently embedded” as used in this description is intended to mean that the parts, permanently connected or permanently embedded, requires the use of tools in order to be separated and should the parts be separated it would permanently damage at least one of the parts thereby rendering the construction useless for its purpose.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 shows an exploded view of the dose engine according to the present invention.

FIG. 2 shows a cross sectional view of the dose engine in FIG. 1 during dose setting.

FIG. 3 shows a cross sectional view of the dose engine in to FIG. 1 during dose expelling.

FIG. 4 shows a cross sectional view of the housing.

FIG. 5 shows a perspective view of the piston rod driver.

FIG. 6A shows a perspective view of the drive element.

FIG. 6B shows a cross sectional view of the drive element in FIG. 6A

FIG. 7A shows a cross sectional view of the ratchet tube.

FIG. 7B shows a side view of the ratchet tube in FIG. 7A.

FIG. 7C shows an enlargement of the distal end of the ratchet tube marked in FIG. 7B.

FIG. 7D show a perspective view of the ratchet tube mounted in the drive element.

FIG. 8 show a side view of the drive assembly according to the present invention.

FIG. 9 show a cross sectional view of the proximal part of the dose engine during dose setting.

FIG. 10 show a cross sectional view of the proximal part of the dose engine during dialling down a set dose.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “anti (or counter) clockwise” or similar relative expressions are used, these only refer to the appended figures and not to an actual situation of use. The shown figures are schematic representations for which reason the configuration of the different structures as well as their relative dimensions are intended to serve illustrative purposes only.

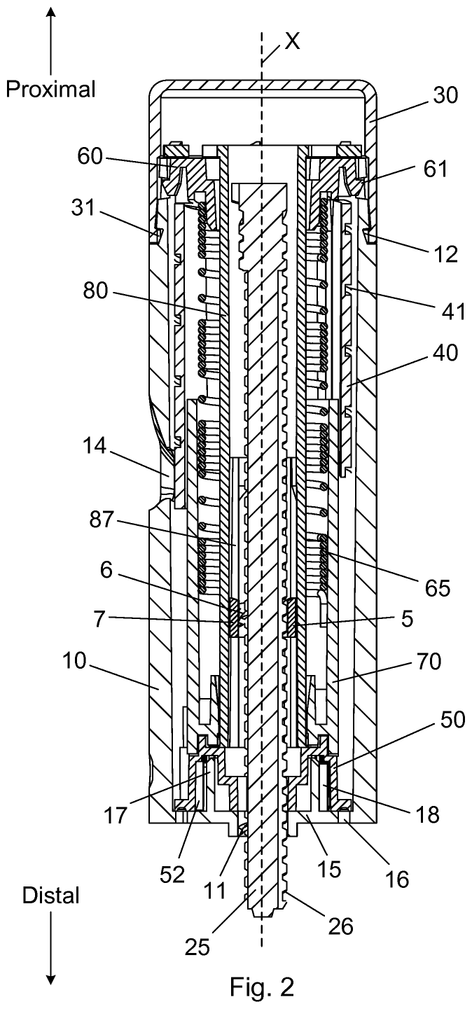

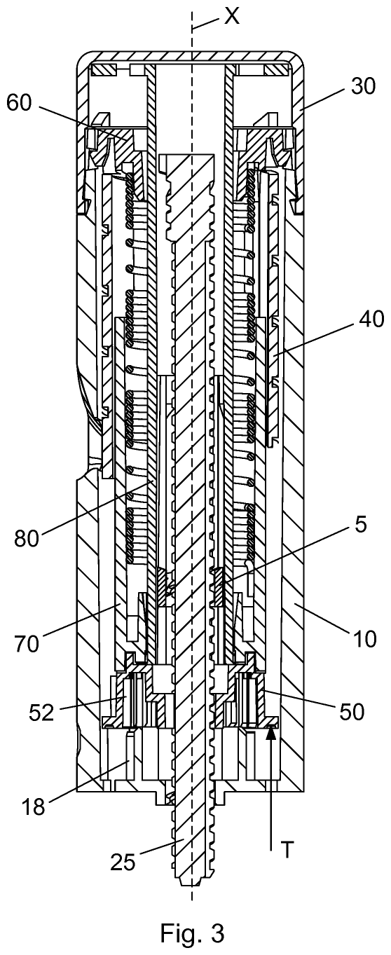

In that context it may be convenient to define that the term “distal end” in the appended figures is meant to refer to the end of the dose setting and injection mechanism device which is usually coupled to the cartridge holder which further carries the injection needle whereas the term “proximal end” is meant to refer to the opposite end pointing away from the cartridge holder and the injection needle and which proximal end usually carries the dose dial button. Distal and proximal is meant to be along an axial orientation of the dose setting and injection mechanism along a virtual centre line marked “X” in FIGS. 2 and 3.

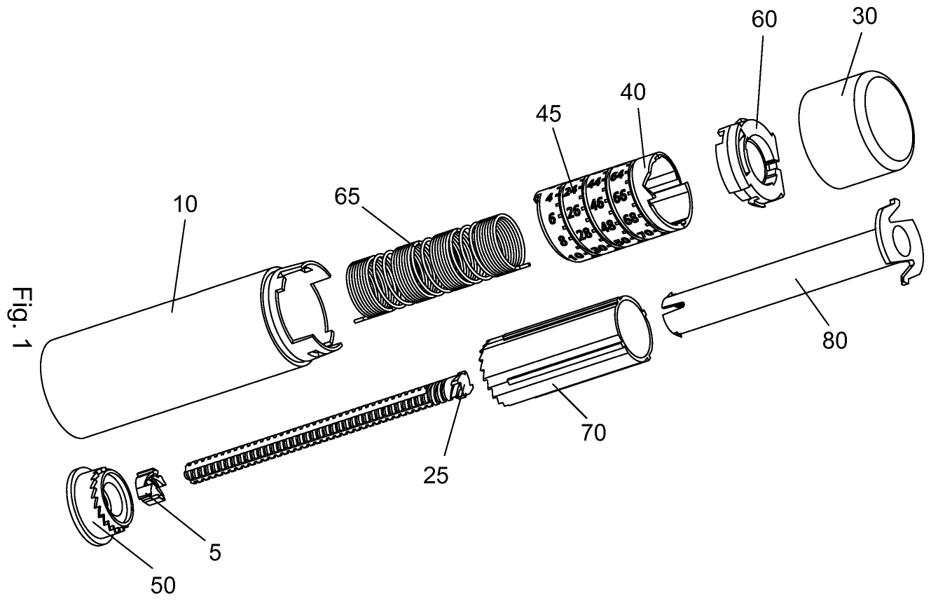

The FIGS. 1 to 3 of the invention disclose the proximal part of the injection device which is often referred to as the dose setting and injection mechanism or simply the dose engine. The housing 10 depicted in FIGS. 1 to 3 is distally coupled to a not shown cartridge holder which secures the cartridge containing the liquid drug to be injected. Together the dose engine and the cartridge holder constitute the injection device.

FIG. 1 further discloses an exploded view of the main parts of the dose engine, which are;
An End-of-Content counter 5.
A Housing 10.
A piston rod 25.
A dose dial 30.
A scale drum 40 (with indicia 45 carried thereon).
A piston rod driver 50.
A spring base 60.
A torsion spring 65 (coiled with open coils 66).
A drive element 70.
A ratchet tube 80.

As best seen in FIGS. 2 and 3, the housing 10 is distally provided with an inner thread 11 in which thread 11 the piston rod 25 operates and can be moved helically forward to press out liquid drug form the not shown cartridge.

The piston rod 25 is on the outer surface provided with an outer thread 26 and a number of longitudinal tracks 27. The outer thread 26 is operated in the inner thread 11 of the housing 10 such that whenever the piston rod 25 is rotated it is moved axially in a helical movement.

At the proximal end, the housing 10 is provided with a dose dial 30 which on the inner surface is provided with a ring-shaped ridge 31 engaging a similar ring-shaped groove 12 provided in the housing 10 such that the dose dial 30 can only be rotated relatively to the housing 10 but is unable to move axially in relation to the housing 10.

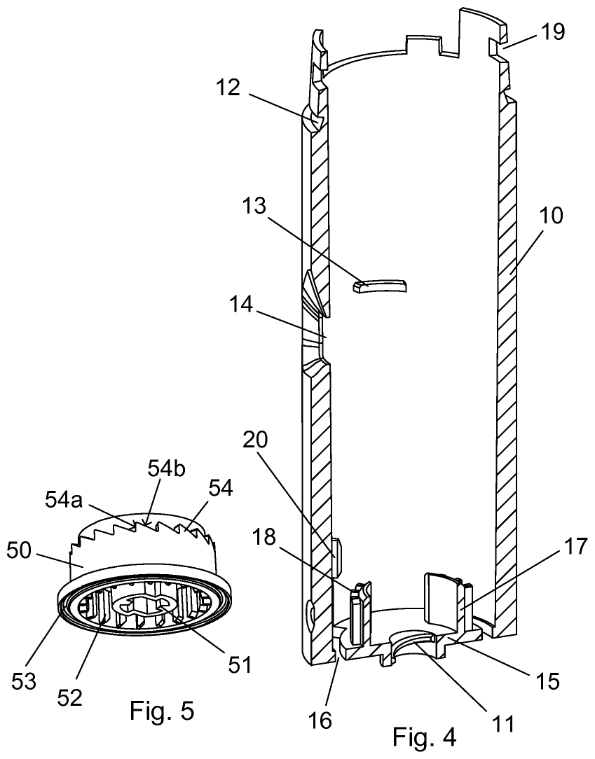

The housing 10 is further illustrated in FIG. 4. The distal end surface 15 of the housing 10 carrying the inner thread 11 is provided with one or more axial openings 16 which relates to the trigger mechanism as will be explained latter. The end surface 15 could alternatively be formed as a separate member secured in the housing 10. Pointing in the proximal direction, the distal end surface 15 is further provided with one or more axially pointing structures 17 which on the outer surface are provided with a number of radially outwardly pointing teeth 18.

The housing 10 is also, preferably on the inner surface, provided with one or more helical protrusions 13 for rotationally guiding the scale drum 40 which is at least partly visible to the user through a window 14 in the housing 10 such that a user can inspect the size of the set dose during dose setting. An inwardly pointing stop protrusion 20 for the scale drum 40 is also provided on the inner surface of the housing 10.

The longitudinal tracks 27 of the piston rod 25 are engaged by a key structure 51 provided on an inner surface of the piston rod driver 50 as disclosed in FIG. 5. This key structure 51 henceforth engages the longitudinal tracks 27 provided on the piston rod 25 such that rotation of the piston rod driver 50 is transformed to a rotation of the piston rod 25 and vice versa. The piston rod 25 and the piston rod driver 50 thus rotate in unison, but can move axially in relation to each other

The piston rod driver 50 is further provided with radially inwardly pointing teeth 52 which engages the radially outwardly pointing teeth 18 provided on the axially pointing structure 17 of the housing. When these two sets of radial teeth 18, 52 are engaged, the piston rod driver 50 is unable to rotate relatively to the housing 10.

Proximally, the piston rod driver 50 is provided with a plurality of V-shaped teeth 54 which engages a drive element 70 as will be explained later.

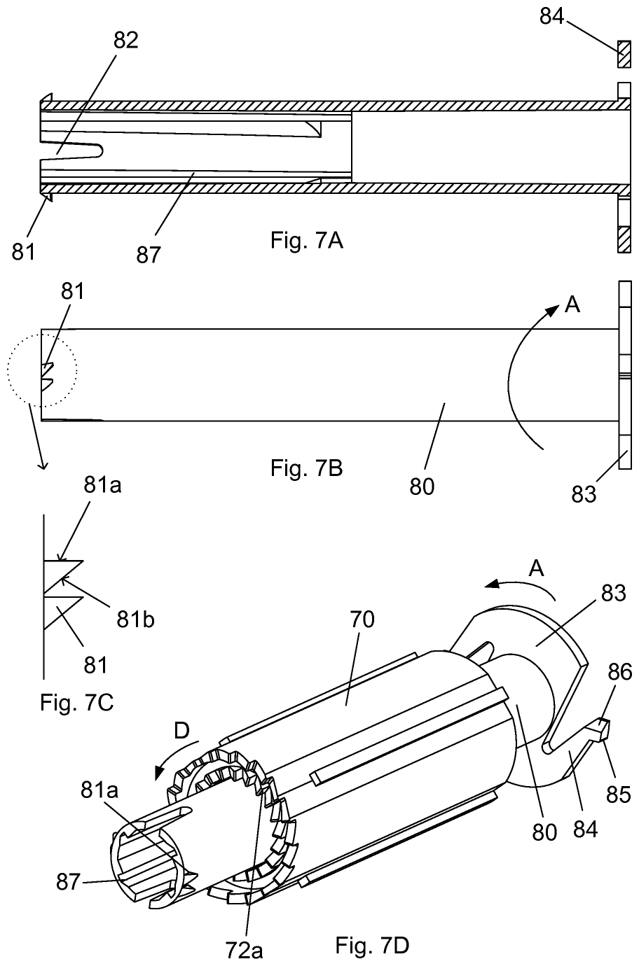

All the V-shaped teeth used in the different engagement in this specification has a steep flange which is referenced with an “a” and a sloped flange which is referenced with a “b”. This is e.g. indicated in FIG. 5 and in FIG. 7C.

The piston rod driver 50 is distally provided with a rim 53 which can be engaged by a release trigger which operates through the axial openings 16 provided in the housing 10. Such release trigger would usually be coupled to a needle shield such that a movement of the needle shield in the proximal direction forces the release trigger to lift the piston rod driver 50 in the proximal direction and out of its engagement with the housing 10.

A similar shield triggered release arrangement is disclosed in WO16/041883 wherein the reference number “20” designates the needle shield and the release arms numbered “23” is the release trigger. The nut holder numbered “60” is the distal part of the housing of the dose engine and is provided with openings “64” through which the release arms “23” operate to move the piston rod driver “55” axially out of engagement with the housing part “60”.

The released position of the present invention is disclosed in FIG. 3 wherein the radially inwardly teeth 52 has been moved out of engagement with the radially outwardly pointing teeth 18 of the housing 10. The direction and action of the release trigger is indicated by the arrow “T” in FIG. 3 which also shows that the release trigger operates through the axial openings 16 in the distal end surface 15 of the housing 10.

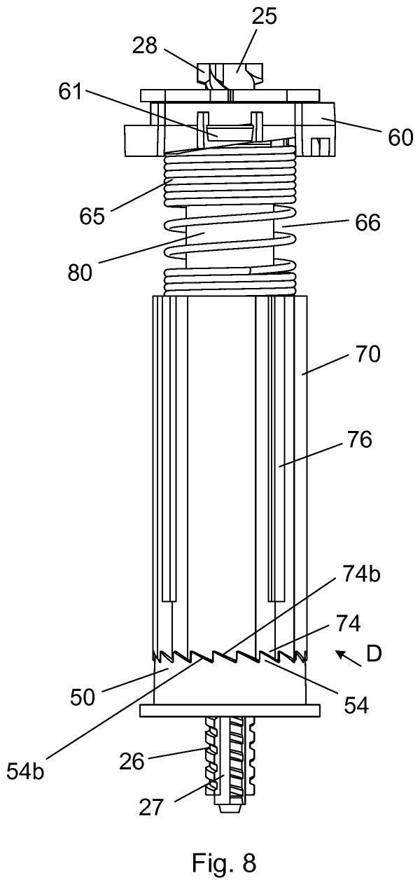

As further disclosed in FIGS. 1 to 3, the housing 10 is towards the proximal end provided with the spring base 60 which is inserted in the housing 10. This spring base 60 has a number of radial teeth 61 (see also FIG. 8) engaging openings 19 in the housing 10 (see e.g. FIG. 4) such that the spring base 60 is both rotationally and axially prevented from moving relatively to the housing 10. Alternatively, the spring base 60 could be moulded as an integral part of the housing 10 and the distal end surface 15 of the housing 10 could be removable to gain access to the interior of the housing 10 during assembly of the dose engine.

The spring base 60 connects to the proximal end of the torsion spring 65 which at its distal end is connected to the drive element 70. The torsion spring 65 is in the disclosed embodiments a torsion spring 65 which applies a rotational torque when rotational strained. The torsion spring 65 is coiled with a distance 66 between at least some of the windings such that the torsion spring 65 also applies an axial force when compressed. The torque accumulated in the torsion spring 65 during dose setting is transformed to a rotation of the drive element 70 during ejection of the set dose.

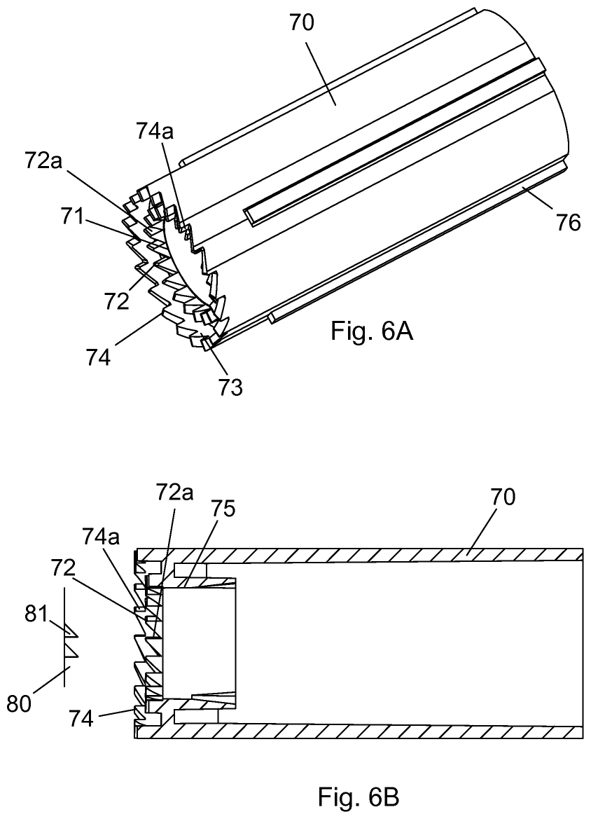

The drive element 70 is disclosed in details in FIG. 6A-B. Distally the drive element 70 is provided with an inner ring 71 of V-shaped teeth 72 and an outer ring 73 of V-shaped teeth 74. Internally the drive element 70 is provided with an inner guide 75 for guiding the ratchet tube 80 as will be explained. On the outside surface of the drive element 70, one or more raised bars 76 are provided which engages similar longitudinal tracks provided in the scale drum 40 such that the scale drum 40 is forced to rotate together with the drive element 70 but is able to slide axially in relation to the drive element 70.

Whenever the drive element 70 rotates so does the scale drum 40, however, since the scale drum 40 engages the helical protrusions 13 provided inside the housing 10, the scale drum 40 travels helically when rotated. The scale drum 40 is on the outer surface provided with a helical track 41 engaging the helical protrusion 13 inside the housing 10 to accommodate the helical movement of the scale drum 40. The outer surface of the scale drum 40 is provided with indicia 45 printed or otherwise provided in a helical row such that the indicia 45 is moved pass the window 14 in the housing 10 upon rotation of the drive element 70 and thus the scale drum 40.

As previously mentioned, the ratchet tube 80 is guided by the inner guide 75 provided inside the drive element 70. The ratchet tube 80 is disclosed in details in FIGS. 7A-C and is distally provided with a number of V-shaped teeth 81. These V-shaped teeth 81 are in the disclosed embodiment provided in pairs approximately 180 degrees separated. The ratchet tube 80 is at its distal end sliced into two halves separated by a longitudinal opening 82 such that the two halves can bend inwardly toward each other during assembly of the injection device.

As seen in FIG. 7C, which depicts an enlarged view of the distal end of the ratchet 80, the V-shaped teeth 81 of the ratchet 80 have a steep flange 81a and a sloped flange 81b. Further, the rotational direction of the ratchet 80 during dose setting is clockwise and indicated by the arrow “A” in FIG. 7B.

The V-shaped teeth 81 on the ratchet tube 80 engages with the V-shaped teeth 72 provided on the inner ring 72 of the drive element 70 as indicated in FIG. 7D. However, as an alternative the drive element 70 and the ratchet tube 80 could be stiffly connected or even moulded as one unitary element, however in such embodiment it would not be possible to make the zero point adjustment explained later.

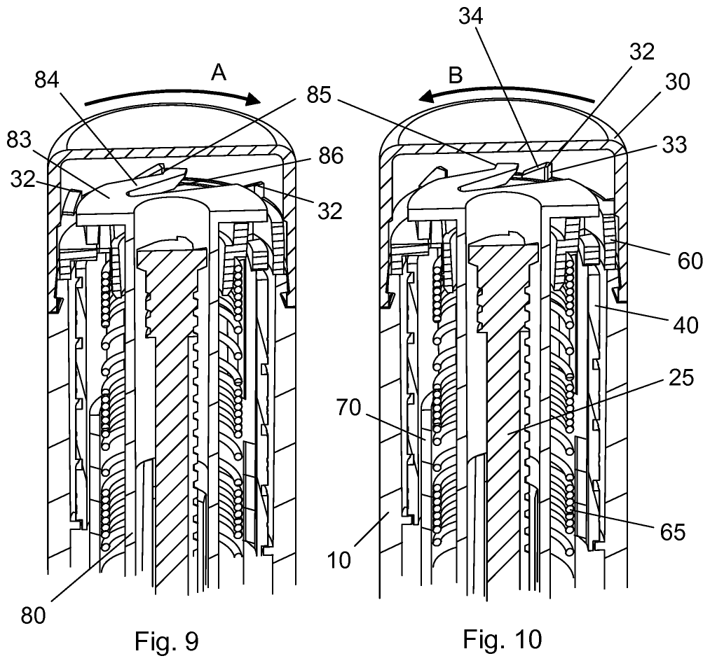

At the proximal end, the ratchet tube 80 is provided with a radially extending flange 83 as best seen in FIG. 7D and in the FIGS. 9-10. This radial flange 83 is further provided with a number of radial arms 84. At the outer tip each of these radial arms 80 carries an axially pointing driving flange 85 and a sloped lifting flange 86. The radial arms 80 can be provided in any random number such as 1, 2, 3 or 4 radial arms 80. In the embodiment of the drawings two radial arms 80 are disclosed.

To operate these radial arms 84, the dose dial 30 is on the inside provided with a number of wedge-shaped protrusions 32 as seen in FIGS. 9 and 10. Each of these wedge-shaped protrusions 32 has an axial driving flange 33 and a sloped lifting flange 34.

FIG. 8 discloses the drive assembly which comprises the spring base 60, the torsion spring 65, the drive element 70 and the piston rod driver 50. FIG. 8 further disclose the ratchet tube 80 and the piston rod 25.

The torsion spring 65 is coiled with one or more open coils 66 such that the torsion spring 65 also applies an axial force between the spring base 60 and the drive element 70. This axial force urges the V-shaped teeth 74 on the outer ring 73 of the drive element 70 into engagement with the V-shaped teeth 54 provided proximally on the piston rod driver 50.

The axial force of the torsion spring 65 and the shape of these V-shaped teeth 54, 74 allow a rotation of the drive element 70 relatively to the piston rod drive 50 in one direction along the sloped flanges 54b, 74b as indicated by the arrow “D” in FIG. 8. As the piston rod driver 50 is rotational locked during dose setting the only allowable rotational direction of the drive element 70 is in the dose setting direction. During this rotation of the drive element 70, the drive element 70 moves axially forth and back a short distance each time the teeth 74 passes over the teeth 54 on the piston drive driver 50. This short axial movement of the drive element 70 occurs against the axial bias of the torsion spring 65.

The steep flanges 54a, 74a prevents the torsion spring 65 from moving the drive element 70 in the counter clockwise direction (against “D”) relatively to the piston rod driver 50. The torque build up in the torsion spring 65 during dose setting is thus maintained stored in the torsion spring 65 due to the engagement of steep flange 74a of the V-shaped teeth 74 with the steep flange 54a of the V-shaped teeth 54 on the piston rod driver 50.

As also seen in FIG. 8, the piston rod 20 is at its proximal end provided with a stop flange 28 which cooperates with and End-of-Content counter 5 as will be explained.

The different operations of the injection device in respect of the embodiment disclosed in the drawings will be explained in the following.

### Dose Setting

In order to set the size of the dose to be ejected, the user rotates the dose dial 30 relatively to the housing 10. The dose setting direction in the described embodiment is clockwise when viewed from the proximal end, hence the user rotate the dose setting button 30 in the clockwise direction as also indicated by the arrow “A” in FIG. 9. The axial driving flange 33 on the wedge-shaped protrusion 32 of the dose dial 30 engages the driving flange 85 on the radial arm 84 of the ratchet tube 80 as seen in FIG. 9 and forces the ratchet tube 80 to follow the rotation of the dose dial 30 in the clockwise direction. Preferably two radial arms 84 are provided whereas the number of wedge-shaped protrusions 32 is somewhat higher.

The steep flange 81a of the V-shaped teeth 81 provided distally on the ratchet tube 80 engages the steep flange 72a on the V-shaped teeth 72 on the inner ring 71 on the drive element 70 such that the clockwise rotation of the ratchet tube 80 during dose setting is transferred to a similar clockwise rotation of the drive element 70. This is best seen in FIG. 7D wherein the ratchet tube 80 has been visually moved out of engagement with the drive element 70. As indicated in FIG. 7D, once the user rotate the ratchet tube 80 in the clock wise direction “A”, the drive element 70 follows in the same direction “D” as also disclosed in FIG. 8.

During the rotation of the dose dial 30 together with the ratchet tube 80 and the drive element 70 in the clockwise direction (“A” in FIG. 9), the sloped flange 74b of V-shaped teeth 74 on the outer ring 73 of the drive element 70 rides over the sloped flange 54b of the V-shaped teeth 54 provided distally on the piston rod driver 50 as the piston rod driver 50 is inrotatable secured in the housing 10. This is also disclosed in FIG. 8.

The dose setting operation is further disclosed in FIG. 2 wherein the radially inwardly pointing teeth 52 on the piston rod driver 50 engages the radially outwardly pointing teeth 18 thereby preventing rotation of the piston rod driver 50. The toothed engagement 18, 52 between the piston rod driver 50 and the housing 10 can also be seen in FIGS. 4 and 5.

The result is thus that the torsion spring 65 encompassed between the drive element 70 and the housing 10 via the spring base 60 is strained and a torque is built up in the torsion spring 65. This torque is maintained in the torsion spring 65 by the engagement between steep flanges 54a on the V-shaped teeth 54 of the piston rod driver 50 and the steep flanges 74a on the V-shaped teeth 74 of the drive element 70. The drive element 70 is thereby prevented form rotation in the counter-clockwise direction.

During dose setting, the scale drum 40 is rotated by the drive element 70 and moved helically such that the increasing size of the set dose is viewable through the window 14.

FIG. 2 discloses the scale drum 40 in the zero position as the scale drum 40 abuts the spring base 60. From this zero position, the user can set a random dose size and the scale drum 40 rotate in the distal direction during dose setting.

### Dose Expelling

Once a dose has been set and the torsion spring 65 is strained, the injection device is ready to eject the set dose as disclosed in FIG. 3. In order to do this, the user presses a not-shown needle shield against the skin of the user. The thereby generated movement in the proximal direction of the needle shield is instantly transferred to a similar axial, and proximal, movement of the transfer element “T”. The transfer element “T” engages the rim 53 on the piston rod driver 50 such that the axial movement is transferred to a similar axial movement of the piston rod driver 50.

The transfer element which is indicated by an arrow marked “T” can either be a separate element moved by the needle shield or it can be an integral part of the needle shield as e.g. known from WO16/041883.

The axial movement of the piston rod driver 50 also moves the drive element 70 proximally against the axial bias of the torsion spring 65 which apply an axial force due to the open distance 66 between at least some of the coils. The ratchet tube 80 follows the axial movement of the drive element 70 since the piston rod driver 50 presses the V-shaped teeth 81 of the ratchet tube 80 into engagement with the V-shaped teeth 72 such that the ratchet tube 80 moves axially in the proximal direction together with the drive element 70.

Following the injection, when the needle shield is removed from the skin of the user, the axial compression of the torsion spring 65 urges the drive element 70, the piston rod driver 50 and the ratchet tube 80 into the initial position disclosed in FIG. 2.

However, in the dose expelling position disclosed in FIG. 3, the radially inwardly pointing teeth 52 on the piston rod driver 50 are moved out engagement with the radially outwardly pointing teeth 18 inside the housing 10 and nothing is preventing the drive element 70 and the piston rod driver 50 from rotating under the influence of the torque of the torsion spring 65.

In the dose expelling scenario disclosed in FIG. 3, the torsion spring 65 applies a rotational torque onto the drive element 70 which therefore rotates. This rotation is via the engagement between the steep flange 74a on the teeth 74 and the steep flange 54a on the teeth 54 transferred to a rotation of the piston rod drive 50. The piston rod drive 50 is internally provided with a key structure 51 which engages the longitudinal tracks 27 provided in the piston rod 25 which is henceforth forced to follow the rotation of the piston rod driver 50. Since the outer thread 26 on the piston rod 25 is threaded to the inner thread 11 in the housing 10, the rotation of the piston rod 25 screws the piston rod 25 in the distal direction in a helical movement. This helical movement of the piston rod 25 in the distal direction presses liquid drug out from the cartridge secured in the injection device.

When the drive element 70 rotate in the counter-clockwise direction during dose expelling, the steep flanges 72a on the drive element 70 transfer rotation to the steep flanges 81a on the ratchet tube 80 with the result that the ratchet tube 80 rotate together with the drive element 70. Since both the drive element 70 and the ratchet tube 80 has been moved proximally by the transfer element “T”, the radial arms 84 of the ratchet tube 80 has been moved out of engagement with the dose dial 30. The result being that the dose dial 30 is decoupled and do not rotate during dose expelling. Further, any rotation of the dose dial 30 executed by the user during dose expelling is not transferred to the ratchet tube 80. The user is thereby prevented from affecting the dose mechanism during dose expelling.

As the drive element 70 rotate in the counter-clockwise direction during dose expelling it transfers this rotation to the scale drum 40 via the splined engagement between the raised bars 76 on the drive element 70 and the scale drum 40.

As the scale drum 40 rotate back to its initial zero position, the indicia 45 helically carried on the scale drum 40 passes by the window 14. At the zero position i.e. when a zero (or similar indicia) is showing in the window 14, the proximal end of the scale drum 40 abut the spring base 60 as e.g. shown in FIGS. 2 and 3.

Conclusively; during dose expelling, the torsion spring 65 rotate the drive element 70 which in turn also rotate the piston rod driver 50, the ratchet tube 70 and the scale drum 40 until the scale drum 40 abuts the spring base 60 in the zero position. The rotation of the piston rod driver 50 causes the piston rod 25 to also rotate and thus be moved in the distal direction.

The spring base 60 could in one example be provided with a flexible arm or the like which could generate a discrete sound once the scale drum 40 abuts the spring base 60 at the end ejection of the set dose.

### Resetting

If the user has set a dose size which is higher than the amount that the user actually wants to inject, the user can reset the set dose by simply rotating the dose setting button 30 in the opposite direction which in the disclosed embodiment is in the counter clockwise direction as indicated by the arrow “B” in FIG. 10.

During this rotation, the lifting flange 34 of the wedge-protrusions 32 inside the dose dial 30 engages the sloped lifting flanges 86 on the radial arms 84 of the ratchet tube 80 such that the ratchet tube 80 is lifted in the proximal direction a distance determined by the angle and the height of the wedge-protrusion 32.

The proximal movement of the ratchet tube 80 is transferred to a similar proximal movement of the drive element 70 due to the engagement between the teeth 81 on the ratchet tube 80 and the V-shaped teeth 72 provided on the inner ring 72 of the drive element 70.

The angle and height of the wedge-shaped protrusions 32 are determined such that the V-shaped teeth 74 on the drive element 70 is lifted out of engagement with the V-shaped teeth 54 on the piston rod driver 50 during the proximal movement of the ratchet tube 80 and the drive element 70. Once the V-shaped teeth 74 are lifted out of engagement with the V-shaped teeth 54 on the piston rod drive 50, the torsion spring 65 rotate the drive element 70 in the counter clockwise direction. However, the axial force in the torsion spring 65 instantly moves the drive element 70 in the distal direction such that the toothed engagement 54, 74 between the V-shaped teeth 74 and the V-shaped teeth 54 is only moved one tooth in the counter clockwise direction.

As a result of this, the set dose is only reduced by one increment each time the ratchet tube 80 is lifted proximally by the wedge-shaped protrusion 32.

### Over-Torque

The disclosed injection device has two different over-torque functions. One when the user keeps rotating the dose dial 30 after the scale drum 40 has reached its maximum position and one if the user keeps rotation the dose dial 30 once the scale drum 40 has reached its zero (minimum) position.

Over-torque in maximum position.

When a user rotate the dose dial 30 in the clockwise direction to set a dose, the scale drum 40 travels helically in the proximal direction. FIGS. 2-3 and FIG. 9-10 all disclose the scale drum 40 positioned in the zero position. Usually in this position a “0” or similar indicia 45 carried on the scale drum 40 will be visible in the window 14 in the housing 10

When the scale drum 40 travels distally from the zero position depicted in FIG. 2, the indicia 45 carried on the scale drum 40 passes by the window 14. Once the maximum dose is reached the scale drum 40 abut the inwardly pointing stop protrusion 20 provided inside the housing 10 as disclosed in FIG. 4. Once the scale drum 40 encounters this stop protrusion 20, the scale drum 40 is prevented from further rotation in its threaded connection 41, 13 with the housing 10.

Due to the splined engagement between the scale drum 40 and the drive element 70 and the engagement (72a, 81a) between drive element 70 and the ratchet tube 80, the ratchet tube 80 is also prevented from further rotation in the clockwise direction.

The ratchet tube 80 can also be prevented from further rotation by the End-of-Content counter 5 as explained later.

A continuous rotation of the dose dial 30 will force the wedge-shaped protrusions 32 on the dose dial 30 to force the radial arms 84 radially inwardly such that the wedge-shaped protrusions 32 can pass by the radial arms 84.

The holding torque of the wedge-shaped protrusion 32 against the driving flange 85 of the radial arms 84 is thus determined by the angle of the driving flange 33 on the wedge-shaped protrusion 32 and the driving flange 85 on the radial arms 84 in combination with the resiliency of the radial arms 84.

Over-torque in zero (minimum) position.

If a user keeps rotating the dose dial 30 in the counter clockwise direction to lower the set dose once the scale drum 40 has reached the zero position abutting the spring base 60 the following will happen.

The zero position of the scale drum 40 is disclosed in e.g. FIG. 2. The scale drum 40 abuts the spring base 60 and cannot rotate or move further in the proximal direction, which will also prevent the drive element 70 from rotating in the counter clockwise direction due to the engagement between the raised bars 76 on the drive element 70 which is splined to the scale drum 40.

The wedge-shaped protrusions 32 on the dose dial 30 will therefore lift the ratchet tube 80 in the proximal direction and pass under the radial arms 84 as the dose dial 30 is rotated in the counter clockwise direction (arrow “B” in FIG. 10). As the ratchet tube 80 is lifted in the proximal direction so is the drive element 70, however neither the ratchet tube 80 nor the drive element 70 can rotate any further in the counter clockwise direction and the drive element will just fall down on the same set of teeth 54, 74 once the wedge-shaped protrusion have passed under the radial arms 84.

### End-of-Content

An End-of-Content (EoC) counter 5 is incorporated between the piston rod 25 and the ratchet tube 80 as e.g. disclosed in FIGS. 2 and 3.

On the inside, this EoC counter 5 has a thread 6 having the same pitch as the outer thread 26 of the piston rod 25 and engaging this outer thread 26.

On the outer surface, this EoC counter 5 is provided with longitudinal splines 7 engaging longitudinal guiding tracks 87 provided internally in the ratchet tube 80 such that the EoC counter 5 follows the rotations of the ratchet tube 80. The longitudinal guiding tracks 87 are only provided in the distal half of the ratchet tube 80 as depicted in FIG. 7A since the EoC counter 5 only operates in this area as will be understood from the following.

All though the longitudinal guiding tracks 87 extend in an axial direction the tracks can have a slightly sloped extension in the longitudinal direction to thereby reduce friction in the EoC system.

During dose setting, the piston rod 25 is prevented from rotation due to the engagement (27, 51) with the piston rod driver 50 which is locked to the housing 10 during dose setting. However, the dose dial 30 and the ratchet tube 80 rotate together during dose setting.

Since the EoC counter 5 rotate together with the ratchet tube 80, the EoC counter 5 is rotated on the outer thread 26 on the piston rod 25 and thereby moved in the proximal direction a distance which correlates to the size of the set dose.

If the user rotates the dose dial 30 and the ratchet tube 80 in the counter clockwise direction to lower the set dose, the EoC counter 5 moves in the distal direction on the piston rod 25 accordingly.

During dose expelling as disclosed in FIG. 3, the torsion spring 65 rotates the driver element 70 and the ratchet tube 80 in the counter clockwise direction. The driver element 70 further rotates the piston rod driver 50 which rotates the piston rod 25.

Since the ratchet tube 80 and the piston rod 25 both rotate with the same rotational speed during expelling of the set dose, the EoC counter 5 remains in the same position on the piston rod 25 during expelling.

The EoC counter 5 henceforth only moves during dose setting and only in the proximal direction, and the position of the EoC counter 5 on the piston rod 25 is at any given time an expression of the accumulated set and expelled doses.

During assembly of the injection device, the EoC counter 5 is located in a position on the piston rod 25 such that the EoC counter 5 engages the stop flange 28 on the piston rod 25 once the injectable content in the cartridge has been accumulated set and expelled.

Thus during dose setting the EoC counter 5 is moved proximally on the piston rod 25 and thus moved closer to the stop flange 28 whereas during dose expelling the piston rod 25 is moved distally with the EoC counter 5 remaining in the same position in relation to the piston rod 25 and especially in relation to the stop flange 28. The distance between the EoC counter 5 and the stop flange 28 is at any time an expression of how much injectable liquid drug remains in the cartridge. It is thus not possible to set a dose larger than the remaining content of the cartridge. Since the piston rod 25 is moved in the distal direction during dose expelling, the EoC counter 5 only operates in the distal half of the ratchet tube 80.

In one example the cartridge e.g. contains insulin sufficient for 3.0 millilitre of insulin. The accumulated distance the EoC counter 5 is allowed to move is thus correlated with the 3.0 millilitre of insulin, such that when the user accumulated has set and expelled a total of 3.0 millilitre of insulin the ratchet tube 80 is prevented from further rotation by the EoC counter 5 which in this situation engages the stop flange 28.

The user is thus at any time prevented from setting a dose larger than the remaining injectable content in the cartridge and the relative position of the EoC counter 5 on the piston rod 25 is continuously an expression of the accumulated quantum of drug set and expelled.

The previously described over torque at maximum position also applies to the mechanism when the ratchet tube 80 is prevented from further rotation due to having reached the End-of-Content.

### Zero Point Adjustment

During assembly of a pre-filled injection device it is often desired to have the piston rod to abut the plunger inside the cartridge to thereby avoid initial priming of the injection device. If a piston rod foot is provided between the plunger and the piston rod, the piston rod foot should abut the plunger inside the cartridge. However, this abutment must be made during the assembly of the injection device without moving the position of the EoC counter otherwise a reduced amount of liquid drug will be available for expelling.

In the disclosed embodiment this zero point adjustment is done as herein described.

First the piston rod driver 50 is lifted out of engagement with the housing 10 as disclosed in FIG. 3. In this position the piston rod driver 50 can rotate independently of the housing 10 and since the piston rod driver 50 engages the piston rod 25, the piston rod driver 50 and the piston rod 25 will rotate together and move the piston rod 25 helically forward in the thread 11 of the housing 10 until the piston rod 25 abuts the plunger (e.g. via the piston rod foot).

However, the distance between the EoC counter 5 and the stop flange 28 on the piston rod 25 must be kept permanent during the zero point adjustment such that the intended injectable volume (or number of increments) remains accessible. This is done by rotating the ratchet tube 80 simultaneously with the piston rod 25 such that the EoC counter 5 remains in its relative position on the piston rod 25 such that the distance between the EoC counter 5 and the stop flange 28 on the piston rod 25 remains constant.

During the zero point adjustment, the scale drum 40 is positioned in the zero position. In which position both the scale drum 40 and the drive element 70 is prevented from counter clockwise rotation since the scale drum 40 abut the spring base 60 as depicted in FIG. 3 and is splined to the driver element 70.

Rotation of the ratchet tube 80 and not the driver element 70 is possible due to the toothed engagement 81, 72 between the ratchet tube 80 and the drive element 70.

When the ratchet tube 80 is rotated in the counter clockwise direction during assembly of the injection device, the sloped flange 81b on the teeth 81 will ride under the sloped flange 72b of the V-shaped teeth 72 of the inner ring 71 on the driver element 70.

The assembly is preferably done such that the dose engine as disclosed in FIGS. 1 to 3 is first assembled in one assembly line. The cartridge holder securing the cartridge containing the liquid drug is preferably assembled in a different line.

Before connecting the cartridge holder to the dose engine, the position of the rubber plunger inside the cartridge is determined where after it is calculated how much the piston rod 25 should be moved forward inside the dose engine in order to obtain no or only very little distance between the piston rod 25 and the rubber plunger inside the cartridge.

The piston rod 25 is henceforth rotated together with the ratchet tube 80 such that the EoC counter 5 maintains in its position on the piston rod 25 thus leaving the distance between the EoC counter 5 and the stop flange 28 intact.

Once the piston rod 25 has been brought to its correct position, the dose engine and the cartridge holder are permanently secured to each other to form the pre-filled injection device.

Some preferred embodiments have been disclosed in the foregoing, but it should be stressed that the invention is not limited to these, but may be embodied in other ways within the subject matter defined in the following claims.

1. A torsion spring driven injection device for delivering set doses of a liquid drug, comprising:
a housing having a longitudinal centre axis (X),
a dose setting structure being rotatable in a first direction relative to the housing for setting the size of the individual doses to be ejected,
a piston rod having an outer surface with an outer thread which extend helically in a longitudinal direction and which outer surface further is provided with a longitudinal extending engagement surface such that the outer surface of the piston rod has a non-circular cross section,
a nut member having an inner thread mating the outer thread of the piston rod and which nut member is stationary in the housing,
a rotatable piston rod driver engaging the longitudinal extending engagement surface of the non-circular cross-section of the piston rod such that the piston rod is moved in an axial direction when the piston rod driver is rotated relatively to the housing,
a drive element coupled to rotationally follow the dose setting structure in the first direction during dose setting,
a torsion spring which is encompassed between the housing and the drive element, such that a torque is built up in the torsion spring during rotation of the dose setting structure and the drive element in the first direction, and
which torque is releasable to rotate the drive element in a second direction during ejection of the set dose, the second direction being rotational opposite to the first direction, and
wherein the drive element rotates relatively to the piston rod driver during dose setting and the drive element and the piston rod driver rotate in unison during ejection of the set dose,
characterized in that, a ratchet engagement is provided between the drive element and the piston rod driver,
wherein ratchet engagement comprises a first set of teeth provided on the drive element engaging a second set of teeth provided on the piston rod driver and wherein the drive element and the piston rod driver are urged into engagement by a spring bias.

2. The torsion spring driven injection device according to claim 1, wherein the piston rod driver is rotationally locked to housing during dose setting.

3. The torsion spring driven injection device according to claim 1, wherein the teeth in the first set and the teeth in the second set are V-shaped with an axial flange extending substantially parallel to the centre axis (X) and a sloped flange forming an angle to the centre axis (X).

4. The torsion spring driven injection device according to claim 3, wherein the sloped flange of the teeth on the drive element rides over the sloped flange of the teeth on the piston rod driver during dose setting.

5. The torsion spring driven injection device according to claim 1, wherein the dose setting structure comprises a dose setting button and a ratchet tube.

6. The torsion spring driven injection device according to claim 5, wherein a structure is provided between the dose setting button and the ratchet tube such that rotation of the dose setting button in the second direction moves the ratchet tube proximally.

7. The torsion spring driven injection device according to claim 6, wherein the structure comprises one or more wedge-shaped protrusions having a lifting flange and being located between the dose setting button and the ratchet tube such that the lifting flange moves the ratchet tube in the proximal direction upon rotation of the dose setting button in the second direction.

8. The torsion spring driven injection device according to claim 6, wherein the ratchet tube is coupled to the drive element such that the drive element moves together with the ratchet tube in the proximal direction thus releasing the engagement between the first set of teeth provided on the drive element and the second set of teeth provided on the piston rod driver.

9. The torsion spring driven injection device according to claim 8, wherein the ratchet tube is permanently coupled to the drive element.

10. The torsion spring driven injection device according to claim 8, wherein the ratchet tube has a toothed engagement with the drive element (70).

11. The torsion spring driven injection device according to claim 10, wherein the ratchet tube distally is provided with a plurality of V-shaped teeth, and the drive element has an inner ring of V-shaped teeth, and wherein the V-shaped teeth on the ratchet tube is positioned such in relation to the teeth on the drive element that the ratchet tube is rotational in relation to the drive element in the second direction.

12. The torsion spring driven injection device according to claim 1, wherein the piston rod driver is moved proximally in relation to the housing to a rotationally unlocked position out of engagement with the housing during ejection of the set dose.

13. The torsion spring driven injection device according to claim 12, wherein the piston rod driver is moved proximally in relation to the housing and out of engagement with the housing by a release trigger (“T”) during injection.

14. The torsion spring driven injection device according to claim 12, wherein the torque of the torsion spring rotates the drive element during ejection of the set dose such that a rotational force is transferred from the axial flanges of the teeth on the drive element to the axial flanges on the teeth on piston rod driver thereby creating a rotation of the piston rod driver.
